(19) United States
(12) Patent Application Publication
(10) Pub. No.: US 2022/0225936 A1
(43) Pub. Date: Jul. 21, 2022

(54) CONTACTLESS VITALS USING SMART GLASSES

(71) Applicants: Donald Lane, Goose Creek, SC (US); Leeann M. Bennett, Goose Creek, SC (US)

(72) Inventors: Donald Lane, Goose Creek, SC (US); Leeann M. Bennett, Goose Creek, SC (US)

(21) Appl. No.: 17/577,695

(22) Filed: Jan. 18, 2022

Related U.S. Application Data
(60) Provisional application No. 63/139,212, filed on Jan. 19, 2021.

(51) Int. Cl. A61B 5/00; A61B 5/01; A61B 5/021; A61B 5/024; A61B 5/145; A61B 5/16; G06F 3/01

(52) U.S. Cl. CPC A61B 5/6803; A61B 5/742; A61B 5/0013; A61B 5/749; A61B 5/01; G06F 3/011; A61B 5/024; A61B 5/14542; A61B 5/4869; A61B 5/165; A61B 5/0077; A61B 5/021

(57) ABSTRACT

A non-transitory computer-readable medium storing a communication relay program including instructions that, when executed by a processor, causes an information processing apparatus connected to an image processing apparatus through a communication interface, to: capture, using smart glasses coupled to a user's head, images of a person, to capture, using one or more sensors coupled to the smart glasses, one or more associated signals from the person. And to calculate vital signs of the person based on the images or signals and display the vital signs to the user.

### RELATED APPLICATIONS

This application claims the benefit of U.S. provisional application No. 63,139,212 filed Jan. 19, 2021 and entitled CONTACTLESS VITALS USING SMART GLASSES, which provisional application is incorporated by reference herein in its entirety.

### TECHNICAL FIELD

This disclosure relates to the use of smart glasses and voice recognition to provide an intuitive interface and method of collecting biometric medical data such as body temperature, blood pressure, O2 saturation, and respiration.

### BACKGROUND ART

Health care providers commonly collect vital sign information of patients in order to assist with diagnosing a patient's present medical condition. The problem with technology utilized as part of gathering biometric medical data such as temperature, blood pressure, O2 saturation, respiration, requires using methodologies that are over 180 years old (e.g., stethoscope, blood pressure cuff, temperature probe, etc.). Using these older methods requires an individual to come into social contact or direct contact with the patient. This increases the likelihood of infection and puts the individual collecting the data at risk due to exposure or harm.

Recently, systems and methods have been developed to utilize a combination of cameras, sensors, and computer algorithms to assist medical providers with taking a patient's vital signs in a way that does not require the provider to physically contact the patient. For example, U.S. Pat. No. 10,376,192 to Nuralogix™ (incorporated herein by reference) provides for a system and method for contactless blood pressure determination. U.S. Pat. No. 10,702,173 to Nuralogix™ (incorporated herein by reference) provides for a system and method for camera-based heart rate tracking. Such camera-based methods of obtaining a patient's vital signs can be improved through the integration of such systems with smart glasses and/or voice activated interfaces.

There is a need for a new methodology of collecting contactless vitals using an augmented reality interface coupled with artificial intelligence software to allow for a new way of taking vitals without bodily contact (lowering risk of transmittable disease or viral infection), or the usage of disposable medical products (temperature probe sleeves, disposable blood pressure cuffs, and gloves). The medical industry has a need to take a vital sign using an alternative methodology and system that will allow users to not have to come into close proximity with someone infected by COVID-19 or any other communicable disease.

When factored in the expenses to maintain current bio-medical equipment and removal of disposable medical waste, a solution to this problem will provide for a safer and cleaner alternative. $128 billion annually is spent on the above-referenced medical items globally. The cost savings in medical equipment alone from such a contactless vitals system and method using smart glasses and voice activation could be up to $98 billion annually.

### SUMMARY

In one aspect, a non-transitory computer-readable medium storing a communication relay program including instructions that, when executed by a processor, causes an information processing apparatus connected to an image processing apparatus through a communication interface, to capture, using smart glasses coupled to a user's head, images of a person. Further, the apparatus is configured to capture, using one or more sensors coupled to the smart glasses, one or more associated signals from the person. Then, the apparatus is configured to calculate vital signs of the person based on the images and/or signals. The vital signs may then be displayed to the user. The images and/or signals may be captured by the user using voice activation.

In another aspect, a system is configured to calculate vital signs using smart glasses configured to be worn on a user's head, a camera coupled to the smart glasses configured to captures images of a person, one or more sensors coupled to the smart glasses configured to measure associated signals from the person, and a computer configured to calculate and have displayed to the user vital signs of the person based on the captured images and/or signals.

In another aspect of the disclosure, provided is a method for calculating vital signs comprising capturing, using smart glasses coupled to a user's head, images of a person; capturing, using one or more sensors coupled to the smart glasses, one or more associated signals from the person; calculating vital signs of the person based on the images and/or signals; and displaying the vital signs to the user.

The present disclosure has been made in view of the above-mentioned circumstances and has an object to provide for contactless vitals using smart glasses and voice activation.

The embodiments disclosed in this application to achieve the above-mentioned object has various aspects, and the representative aspects are outlined as follows. With parenthetical reference to the corresponding parts, portions, or surfaces of the disclosed embodiment, merely for the purposes of illustration and not by way of limitation, the present disclosure provides a system and method for capturing and displaying vital signs of a person without physical contact using smart glasses, computer processing, and voice activation.

According to the above noted aspects, the disclosed system and methods provide for a lower cost to maintain equipment and systems, less risk of communicable disease or infection, less medical waste, and a cleaner and greener environment, and also greater protection for medical staff and/or end users of vital sign detection equipment and services.

### BRIEF DESCRIPTION OF THE DRAWINGS

FIG. 1 is a picture of smart glasses configured to perform contactless vitals according to one embodiment of the disclosure.

FIG. 2 is an overhead view of the smart glasses shown in FIG. 1.

FIG. 3 is a picture of a thermal camera used in one embodiment of the disclosure.

FIG. 4 is a flowchart showing the performance of contactless vitals using smart glasses according to one embodiment of the disclosure.

FIG. 5 is a flowchart showing the performance of contactless vitals using smart glasses according to another embodiment of the disclosure.

FIG. 6 is a screenshot of a touch screen interface according to one embodiment of the disclosure.

FIG. 7 is a screenshot of a touch screen interface according to another embodiment of the disclosure.

FIG. 8 is a screenshot of a touch screen interface according to yet another embodiment of the disclosure.

FIG. 9 is a screenshot of a voice activation settings menu according to one embodiment of the disclosure.

FIG. 10 is another screenshot of a voice activation settings menu according to one embodiment of the disclosure.

FIG. 11 is a screenshot of an exemplary smart glasses interface according to one embodiment of the disclosure.

FIG. 12 is an exemplary picture of a doctor wearing smart glasses and taking contactless vitals of a patient, according to one embodiment of the disclosure.

FIG. 13 is a screenshot of a smart glasses interface performing contactless vitals on a patient, integrated with Nuralogix™ software.

FIG. 14 is a screenshot of a Nuralogix™ software interface using vitals obtained via smart glasses according to one embodiment of the disclosure.

FIG. 15 is another screenshot of a Nuralogix™ software interface using vitals obtained via smart glasses according to one embodiment of the disclosure.

### DESCRIPTION OF THE PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read together with the specification and are to be considered a portion of the entire written description of this invention.

The present invention relates to the use of smart glasses, such as Google Glasses EE2 (as shown in FIG. 1), to provide an intuitive interface and alternative method of collecting and determining biometric medical data such as body temperature, blood pressure, O2 saturation, respiration, and the like. The processing of images and/or signals obtained from cameras and/or sensors can be performed by computer software such as that developed by NuraLogix Corporation.

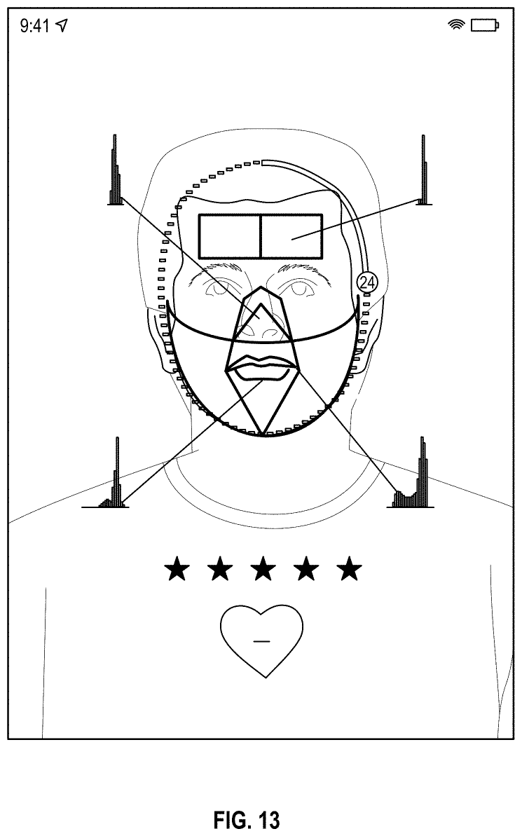

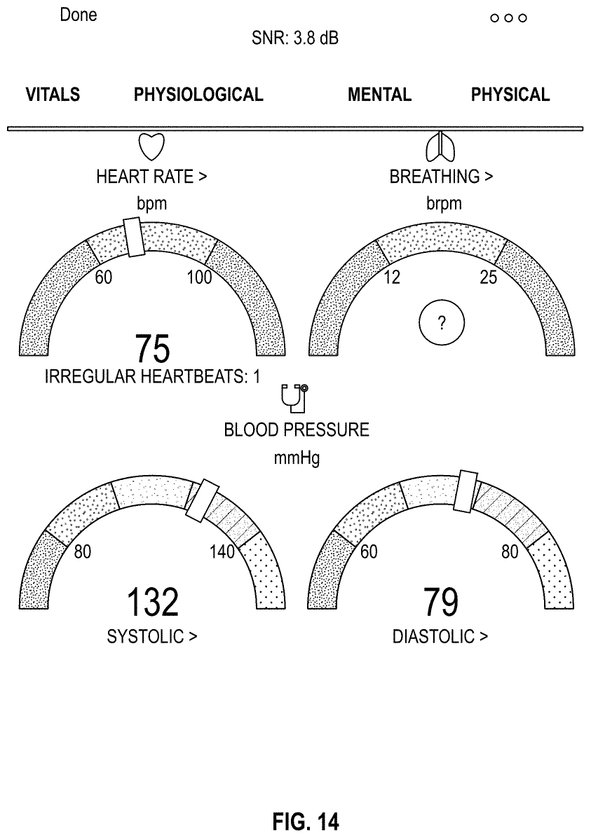

For example, research has been conducted along with a partnership with Nuralogix Corporation to adapt their current Anura SDK app (shown via screenshots in FIGS. 13-15) for scaling in a smart glasses interface to extrapolate biometric (contactless vital sign) data. Further, the application interface has been configured for viewing in landscape mode versus a locked-in angle of portrait mode.

Such software can be enhanced by providing proprietary voice recognition software to allow medical staff or an individual to take a patient's vital signs without coming into direct contact with the patient, allowing the medical professional to remain at recommended social distances. This lowers the likelihood of transmission, risk of exposure to unwanted or highly infectious contaminants that the patient may have. Alternatively, such systems and methods permit medical professional to obtain vital signs remotely (e.g., in a tele-health environment), permit a non-medical person to take vital signs of a patient, and/or permit a patient to take their own vital signs.

In one embodiment of the disclosure, proprietary software code and methods leveraging Google voice open-source code and software development kit (SDK) is disclosed herein. The system is configured to implement a voice recognition feature in combination with smart glasses, which allows for a hands-free experience for use by the wearer of the glasses. The software code may be hosted remotely, or natively in the smart glasses.

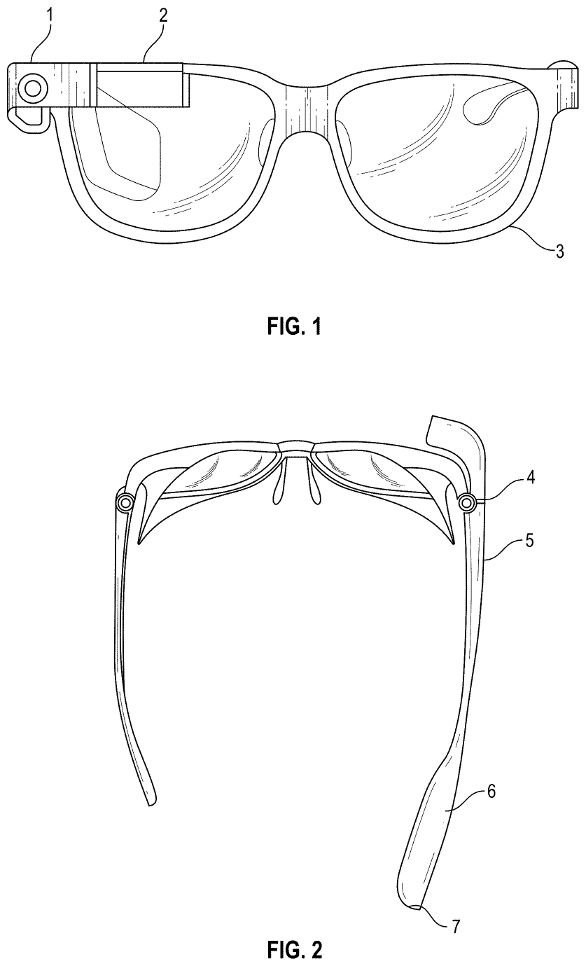

According to one embodiment of the disclosure, the hardware devices shown in FIGS. 1-3 can be configured to provide for obtaining contactless vital signs of a person using voice activation. Initial testing comprised of finding a suitable (small factor) thermal camera that can be connected via Type C connector to the smart glasses physically.

Preferred hardware may comprise any suitable smart glasses system, such as Google Glasses EE2, which is a wearable pair of glasses that utilizes an augmented reality interface and display. Software hosted on such smart glasses may be specifically designed for operation on the smart glasses operating system, such as the Android operating system on Google Glasses EE2.

Suitable software programs for facilitating the processing of biometric data via analysis of collected images or signals for the estimation and display of contactless vitals, such as the artificial intelligence analysis software patented and licensed by NuraLogix Corporation, may be used. However, any suitable image or signal processing software may be used with the disclosed system and methods.

According to one embodiment of the disclosure, Google Glasses EE2 provide an intuitive interface and alternative method of collecting biometric medical data. Utilizing signal processing such as the NuraLogix software in combination with the voice recognition methods disclosed herein, allows medicals staff or an individual to take a patient's vitals without coming into direct contact and allows them to remain at recommended social distances. This lowers the likelihood of transmission, risk of exposure to unwanted or highly infectious contaminants that the patient has.

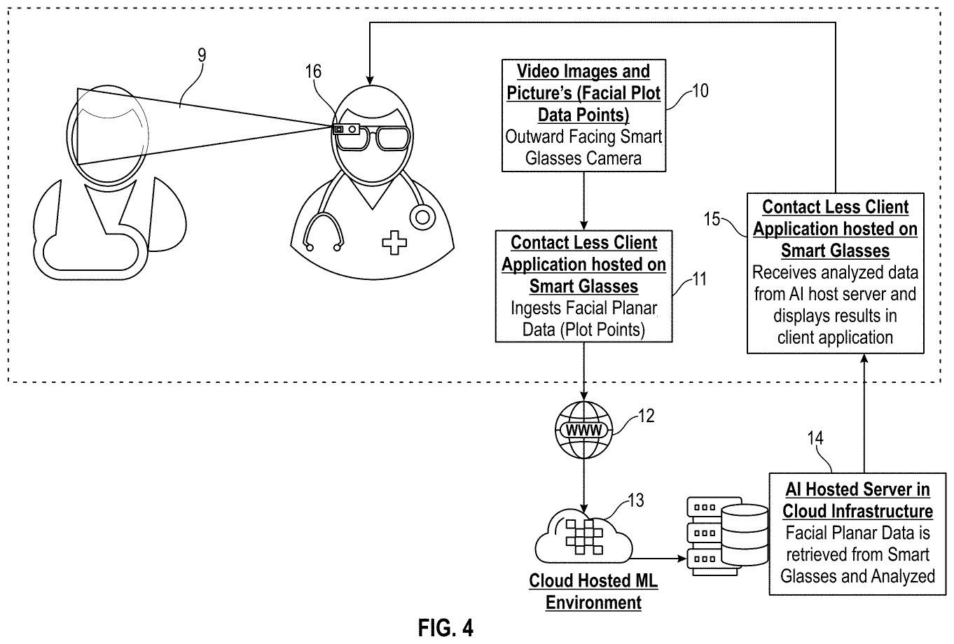

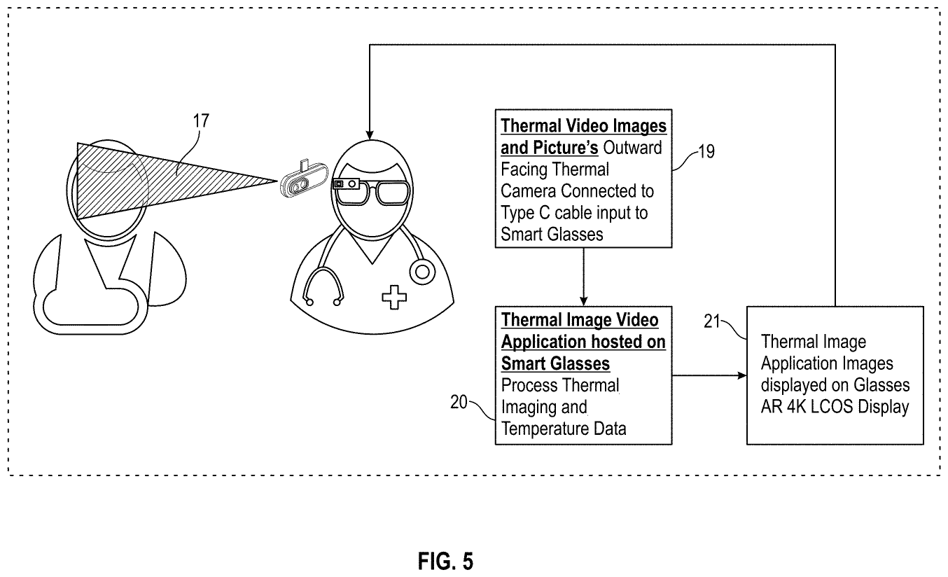

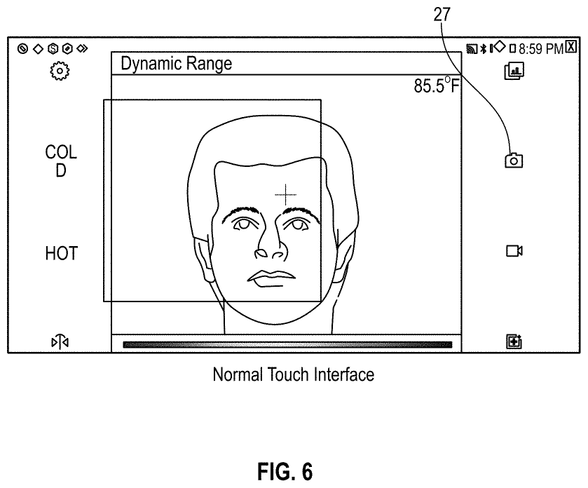

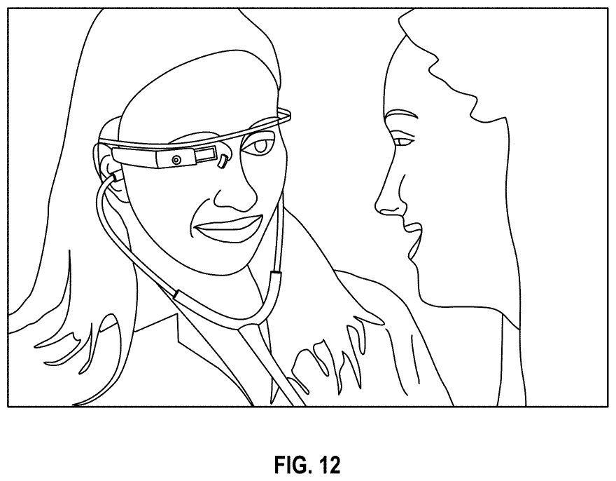

Turning to FIGS. 4-5, a preferred data flow for collecting, analyzing, and displaying contactless vitals is disclosed. FIG. 4 shows an example method and system for collecting vital signs using images obtained from a camera feed. Vital signs that can be estimated and collected via image analysis include blood pressure, heart rate, body mass index, O2 saturation, age, and stress level. Images may be collected via a 4k camera feed (9) using a camera coupled to smart glasses (16). Image processing software may be configured to collect and process specific data points (10), for example facial planar data points. Such data points may be processed using a client-side software application hosted on glasses (11), either alone or in combination with software hosted remotely, such as an AI system hosted on a server (14), which connects to the smart glasses interface (11) via internet (12) and/or a secure cloud environment (13). The AI system (14) may process facial planar data alone or in combination with an application (15) hosted on the glasses. Once the desired vital sign information is obtained through the software system, such vitals are displayed on an augmented reality interface, such as a hi-max LCOS display (16) for the smart glasses wearer. Example interface displays are shown in FIGS. 6-8, 11, and 13.

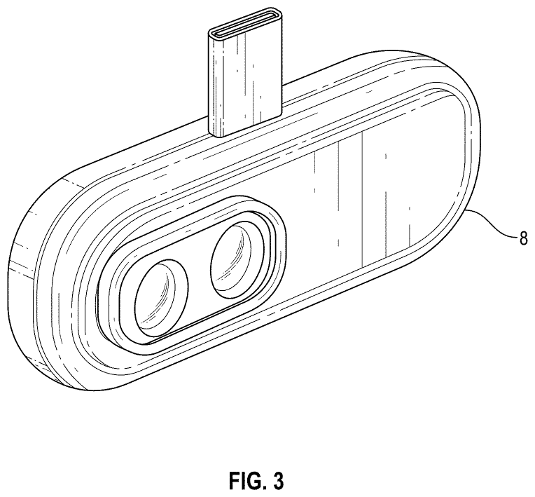

FIG. 5 shows another preferred data flow for collecting signals from a user via smart glasses in order to process and display vital sign information. As shown in FIG. 5, a thermal camera feed and images (17) are obtained using, e.g., micro thermal camera (18). Such thermal images are captured by the thermal camera (example shown in FIG. 3), which may be connected to smart glasses via an interface such as USB-C. A client-side or remotely hosted software application (20) is configured to process thermal image and/or temperature data, similar to how image data is collected and processed and described with reference to FIG. 4. Processed thermal images and temperature data may be displayed on the smart glasses AR 4k LCOS display (21).

The processing of data feeds received from a thermal camera can be accomplished, e.g., by integrating software that is native to the thermal camera with the software program(s) disclosed herein. For instance, an API can be configured to extract thermal camera image feeds from a thermal camera software application and pass along such feeds for further processing (e.g., to a server containing Nuralogix™ software for analysis of the feeds to calculate vital sign information).

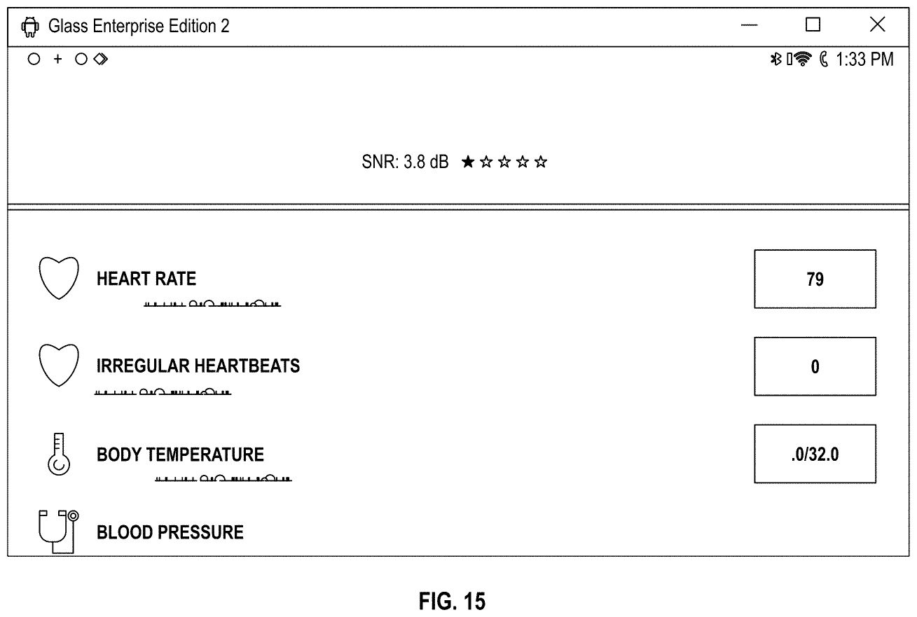

Alternatively, the disclosed system can, for example, be configured to utilize an infrared sensor from a thermal camera to obtain vital sign information (such as body temperature) in a more direct fashion. As illustrated by the application interface screenshot in FIG. 15, the smart glasses can be configured to pull any desired thermal image feed information directly from an infrared sensor via a thermal camera device driver, and send this information along for processing (e.g., by a Nuralogix™ or similar artificial intelligence application). In this way, the disclosed system provides for the processing of metric data for body temperature directly into by a vital sign analysis application without the use of third-party thermal camera software. Thus, API programming requirements may be reduced or eliminated, saving time and costs to get an initial instance of the disclosed system up and running.

FIG. 6 shows am example screenshot of an application interface. In one embodiment, a user may be required to interact with smart glasses via a touch pad (e.g., by tapping a camera icon), will permit the application to take a snapshot for a processing and display of temperature reading.

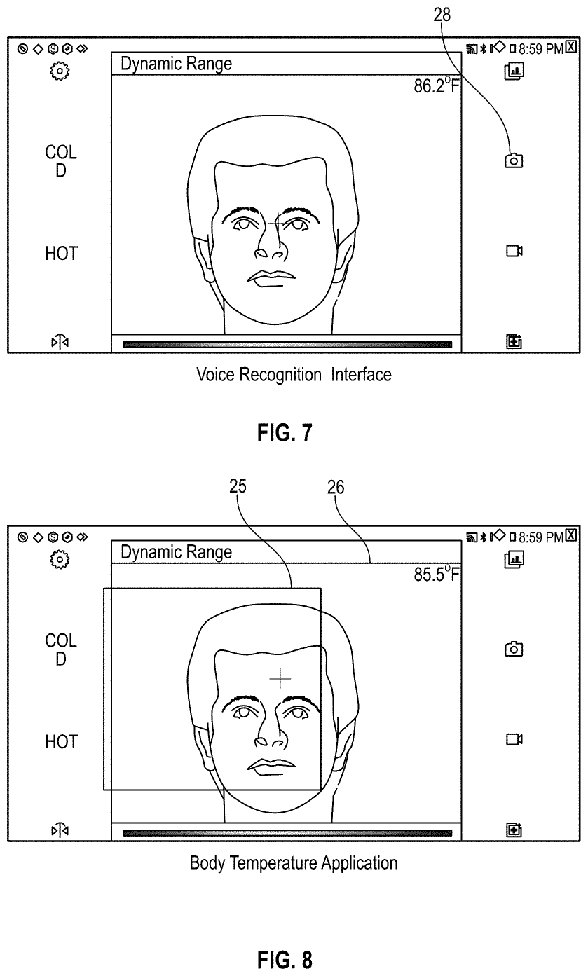

Alternatively, the interface may be configured to impose numerical values to key mapped buttons or icons for any smart glasses' application, wherein the smart glasses wearer can call out the number to activate using his/her voice. E.g., as shown in FIG. 7, a wearer can say the number “four” and the application can be configured to take a snapshot for the temperature processing (28) in the manner described with reference to FIG. 6 above.

As shown in FIGS. 6-8 and 11, processed and displayed vital signs may include heart rate (22), elapsed time for a 30-second scan (23), facial planar data plot points (24), thermal sensor reading point (cross hairs) and/or facial recognition zone (square) (25), and/or temperature surface reading (shown in degrees F.) (26).

With reference to FIGS. 1-3, preferred hardware components according to one embodiment of the disclosure may include a 16-megapixel 4k high-definition camera (1), an augmented reality himax HX7309 LCOS display (2), and safety frames (3), such as OSHA approved safety frames with lenses certified under the ANSI Z87.1-2010 standard. The smart glasses can also be used as part of medical face shield precautions for wearer when taking vitals.

Additional preferred hardware components include a pressure sensitive camera button (4), a gesture control such as a SWIPE 9-degree axis pad (5), smart glasses central CPU compartment (6), a USB type C (or similar) connection (7), and thermal imaging camera (8). In one embodiment, a thermal imaging camera may connect to the smart glasses via a USB type-C connection.

In one preferred embodiment, a contactless vitals client application has been developed for Android OS in landscape mode only, for specific dimensions such as 640×360. Such a configuration may be required to permit the contactless vitals image or signal processing software to properly obtain and analyze preset plot points and send back the proper facial planar data to the AI cloud server for analysis. If the plot points are in error due to client application dimensions that are not properly configured, the vitals may be less accurate than what could be obtained with properly configured application dimensions.

In another preferred embodiment, Google voice recognition settings have been modified in a voice recognition software application to map a specific camera button (4) to turn on or off the voice recognition feature of the smart glasses. Voice recognition allows for hands free operation of the camera and video. Camera operation is still available through multi-touch sensor pad or voice recognition but no longer set to factory default settings from embedded firmware.

According to the preferred disclosure, a user the client application is configured to read body temperatures within 98% accuracy from distance of 1′-18′ away.

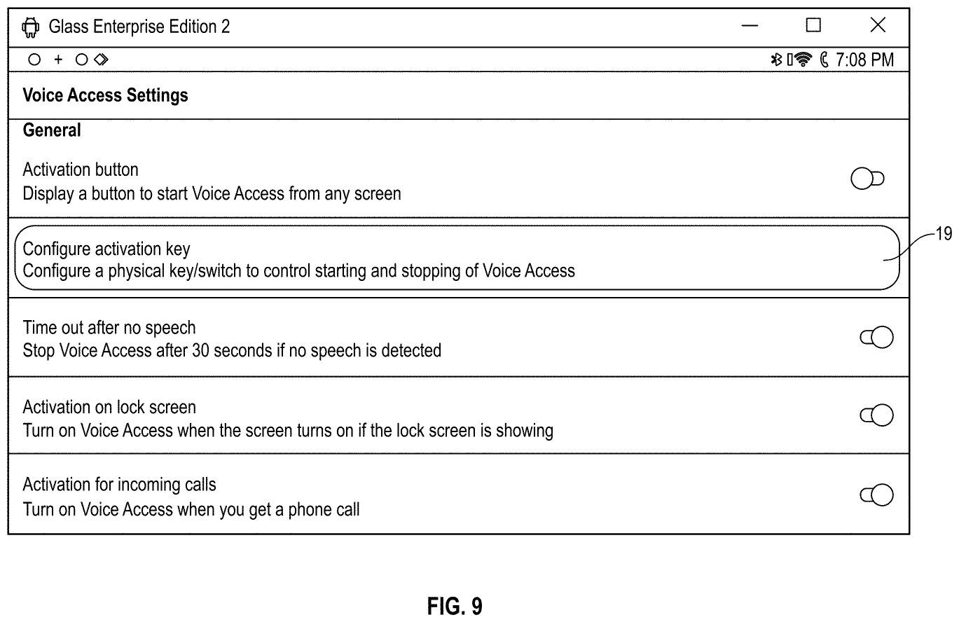

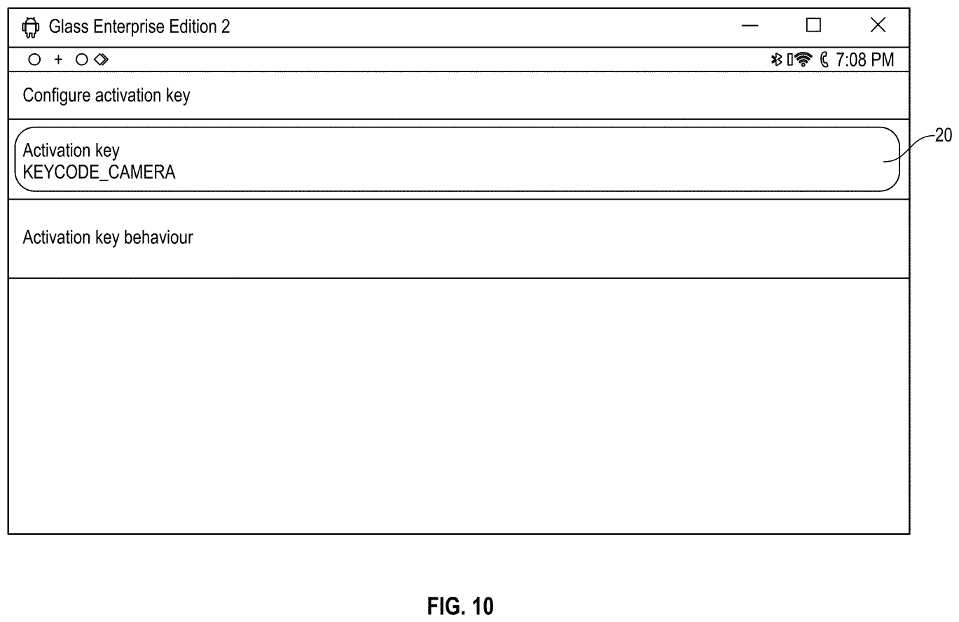

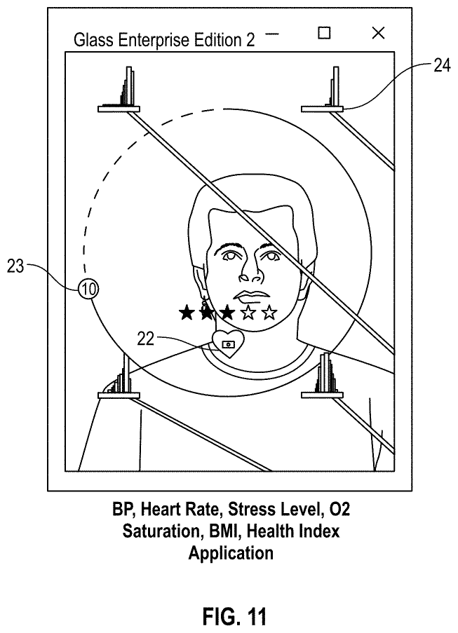

Turning to FIGS. 9-10, shown is a preferred embodiment of an application settings screen for configuring voice activation settings (19), permitting a voice activation key can be mapped to a particular smart glasses button (20). For example, a camera button from Google Glass can be remapped to allow wearer to press and turn on or off for voice recognition.

Exemplar Software Code Key Event and Key Mapping

Key Event Reference:

https://developer.android.com/reference/android/view/KeyEvent

Constants:

“int KEYCODE_CAMERA
Key code constant Camera key.”

Key Mapping of KEYCODE_CAMERA for Voice Activation:

driver.pressKey(keyEvent);
driver.pressKey(new KeyEvent(AndroidKey.CAMERA);
new
KeyEvent(AndroidKey.VoiceAccess).withMetaModifier(KeyEventModifier.
Shift_on);
driver.longPressKey(new KeyEvent(AndroidKey.CAMERA));

One or more of the above example embodiments may be embodied in the form of a non-transitory computer readable medium including thereon computer readable instruction which can be run in a computer through various elements. Examples of the non-transitory computer-readable medium include magnetic media (e.g., hard disks, floppy disks, and magnetic tapes), optical media (e.g., CD-ROMs and DVDs), magneto-optical media (e.g., floptical disks), and hardware devices specifically configured to store and execute program commands (e.g., ROMs, RAMs, and flash memories).

The various functions, processes, methods, and operations performed or executed by the system can be implemented as programs that are executable on various types of processors, controllers, central processing units, microprocessors, digital signal processors, state machines, programmable logic arrays, and the like. The programs can be stored on any computer-readable medium for use by or in connection with any computer-related system or method. Programs can be embodied in a computer-readable medium for use by or in connection with an instruction execution system, device, component, element, or apparatus, such as a system based on a computer or processor, or other system that can fetch instructions from an instruction memory or storage of any appropriate type.

Meanwhile, the computer readable instructions may be specially designed or well known to one of ordinary skill in the computer software field. Examples of the computer readable instructions include mechanical code prepared by a compiler, and high-level languages executable by a computer by using an interpreter.

The methods, programs codes, and instructions described herein and elsewhere may be implemented on or through mobile devices. The mobile devices may include navigation devices, cell phones, smart phones, mobile personal digital assistants, laptops, palmtops, netbooks, pagers, electronic books readers, music players and the like. Computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The particular implementations shown and described herein are illustrative examples of the disclosure and are not intended to otherwise limit the scope of the disclosure in any way. For the sake of brevity, conventional electronics, control systems, software development, and other functional aspects of the systems (and components of the individual operating components of the systems) may not be described in detail. Furthermore, the connecting lines or connectors shown in the various figures presented are intended to represent exemplary functional relationships and/or physical or logical couplings between the various elements. It should be noted that many alternative or additional functional relationships, physical connections, or logical connections may be present in a practical device. Moreover, no item or component is essential to the practice of the disclosure unless the element is specifically described as “essential” or “critical”.

The use of the terms “a”, “an”, “the”, and similar referents in the context of describing the disclosure (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Finally, the steps of all methods described herein can be performed in any suitable order unless otherwise indicated herein or otherwise clearly contradicted by context. The use of any and all examples, or exemplary language (e.g., “such as”) provided herein, is intended merely to better illuminate the disclosure, and does not pose a limitation on the scope of the disclosure unless otherwise claimed. Numerous modifications and adaptations will be readily apparent to those of ordinary skill in this art without departing from the spirit and scope of the present disclosure.

The illustrative block diagrams and flow charts depict process steps or blocks that may represent modules, segments, or portions of code that include one or more executable instructions for implementing specific logical functions or steps in the process. Although the particular examples illustrate specific process steps or acts, many alternative implementations are possible and commonly made by simple design choice. Acts and steps may be executed in different order from the specific description herein, based on considerations of function, purpose, conformance to standard, legacy structure, and the like.

GPS-based position recognition technology, cell location-based position recognition technology, Wi-Fi-based position recognition technology, etc. may be used in the position tracking of the user. However, embodiments are not limited thereto.

The term “camera” refers to a non-contact device designed to detect at least some of the visible spectrum, such as a video camera with optical lenses and CMOS or CCD sensor. The term “thermal camera” refers to a non-contact device that measures electromagnetic radiation having wavelengths longer than 2500 nanometer (nm) and does not touch its region of interest (ROI). A thermal camera may include one sensing element (pixel), or multiple sensing elements that are also referred to herein as “sensing pixels”, “pixels”, and/or focal-plane array (FPA). A thermal camera may be based on an uncooled thermal sensor, such as a thermopile sensor, a microbolometer sensor (where microbolometer refers to any type of a bolometer sensor and its equivalents), a pyroelectric sensor, or a ferroelectric sensor.

A reference to a “camera” herein may relate to various types of devices. In one example, a camera may be a visible-light camera. In another example, a camera may capture light in the ultra-violet range. In another example, a camera may capture near infrared radiation (e.g., wavelengths between 750 and 2000 nm). And in still another example, a camera may be a thermal camera.

The phrase “smart glasses” refers to any type of a device that resembles eyeglasses, and includes a frame configured to be worn on a user's head and includes electronics to operate one or more sensors. The frame may be an integral part of the smart glasses, and/or an element that is connected to the smart glasses. Examples of smart glasses include: any type of eyeglasses with electronics (whether prescription or Plano), sunglasses with electronics, safety goggles with electronics, sports goggle with electronics, augmented reality devices, virtual reality devices, and mixed reality devices. In addition, the term “eyeglasses frame” refers to one or more of the following devices, whether with or without electronics: smart glasses, prescription eyeglasses, Plano eyeglasses, prescription sunglasses, Plano sunglasses, safety goggles, sports goggle, an augmented reality device, virtual reality devices, and a mixed reality device.

Sentences in the form of “a frame configured to be worn on a user's head” or “a frame worn on a user's head” refer to a mechanical structure that loads more than 50% of its weight on the user's head. For example, an eyeglasses frame may include two temples connected to two rims connected by a bridge; the frame in Oculus Rift™ includes the foam placed on the user's face and the straps; and the frame in Google Glass™ is similar to an eyeglasses frame. Additionally, or alternatively, the frame may connect to, be affixed within, and/or be integrated with, a helmet (e.g., a safety helmet, a motorcycle helmet, a combat helmet, a sports helmet, a bicycle helmet, etc.), goggles, and/or a brainwave-measuring headset.

The above-described method for controlling smart glasses may be written as computer programs and may be implemented in digital microprocessors that execute the programs using a computer readable recording medium. The method for controlling the smart glasses may be executed through software. The software may include code segments that perform required tasks. Programs or code segments may also be stored in a processor readable medium or may be transmitted according to a computer data signal combined with a carrier through a transmission medium or communication network.

Embodiments provide for smart glasses capable of taking and analyzing a front image and an image of user's eyes and providing information about a front object selected by user's gaze based on the result of an analysis.

Embodiments also provide for smart glasses capable of analyzing an image of user's eyes and executing a specific function corresponding to user's eye gesture recognized based on the result of an analysis.

In one embodiment as broadly described herein, smart glasses may include a glass having a transparent display function, a first camera configured to obtain a front image, a second camera configured to obtain an image of user's eyes, and a controller configured to analyze the front image and the image of the user's eyes, determine a specific object selected by user's gaze among objects included in the front image based on the result of an analysis, obtain information about the specific object, and display the information about the specific object on a transparent display area of the glass.

The smart glasses may further include a memory configured to store information, and a wireless communication unit connected to a predetermined wireless network. The controller may be connected to the memory or the predetermined wireless network and may obtain the information about the specific object.

The controller may further display a graphic object, indicating that the specific object is selected by the user's gaze, on the transparent display area of the glasses, so that the graphic object is matched with the specific object seen by the user through the glasses.

The controller may display a function list, which is previously determined based on attributes of the selected object, on the transparent display area of the glasses. The controller may execute a function selected by the user's gaze in the function list.

When it is recognized that the user gazes at one edge of the glasses, the controller may rotate the first camera in a direction of the one edge of the glasses and may display an image taken with the rotated first camera on the transparent display area of the glasses.

When it is recognized that the user gazes at one edge of the glasses gaze times, which is previously determined, for a previously determined period of time, the controller may rotate the first camera in a direction of the one edge of the glasses and may display an image taken with the rotated first camera on the transparent display area of the glass.

In another embodiment, smart glasses may include glasses having a transparent display function, a first camera configured to obtain a front image, a second camera configured to obtain an image of user's eyes, and a controller configured to analyze the front image and the image of the user's eyes, execute a specific function corresponding to user's specific eye gesture when the user's specific eye gesture is recognized as the result of an analysis, and display an execution result of the specific function on a transparent display area of the glasses, wherein the controller performs an item selection function included in the execution result of the specific function based on a gesture using user's finger recognized as the result of an analysis of the front image or user's gaze recognized as the result of an analysis of the image of the user's eyes.

When the user's specific eye gesture is recognized, the controller may display an application icon list on the glasses. Further, the controller may execute an application corresponding to an icon selected from the application icon list based on the gesture using the user's finger or the user's gaze or the user's voice and may display the execution result on the glasses.

When an eye gesture, in which the user gazes at a specific area of the glasses, is recognized, the controller may perform a function for displaying previously determined information about the eye gesture on the transparent display area of the glasses.

When the eye gesture, in which the user gazes at the specific area of the glasses, is recognized, the controller may perform a function for displaying system information of the smart glasses on the transparent display area of the glasses.

Smart glasses as embodied and broadly described herein may take and analyze the front image and the image of the user's eyes and may provide information about the specific object selected by the user's gaze based on the result of an analysis.

Smart glasses as embodied and broadly described herein may analyze the image of the user's eyes and may execute the specific function corresponding to the user's eye gesture recognized based on the result of an analysis.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to affect such feature, structure, or characteristic in connection with other ones of the embodiments.

The present disclosure contemplates that many changes and modifications may be made. Therefore, while the presently preferred form of the system has been shown and described, and several modifications and alternatives discussed, persons skilled in this art will readily appreciate that various additional changes and modifications may be made without departing from the spirit of the invention, as defined, and differentiated by the following claims.

What is claimed is:

1. A non-transitory computer-readable medium storing a communication relay program including instructions that, when executed by a processor, causes an information processing apparatus connected to an image processing apparatus through a communication interface, to:
capture, using smart glasses coupled to a user's head, images of a person;
capture, using one or more sensors coupled to the smart glasses, one or more associated signals from the person;
calculate vital signs of the person based on the images or signals; and
display the vital signs to the user.

2. The non-transitory computer-readable medium of claim 1, further configured to permit the capture of the images and/or signals by the user via voice activation.

3. The non-transitory computer-readable medium of claim 1, further configured to permit the calculation and display of vital signs via voice activation.

4. The non-transitory computer-readable medium of claim 1, wherein the vital signs are of the person are displayed to the user via an augmented reality interface on the smart glasses.

5. The non-transitory computer-readable medium of claim 1, wherein the vital signs to be calculated include body temperature, blood pressure, heart rate, O2 saturation, body mass index, age, or stress level.

6. The non-transitory computer-readable medium of claim 1, wherein the smart glasses are the Google™ Glasses EE2.

7. The non-transitory computer-readable medium of claim 1, wherein the calculation of vital signs is performed using the Anura™ software application by NuraLogix™ Corporation.

8. The non-transitory computer-readable medium of claim 1, wherein the smart glasses includes a thermal camera.

9. The non-transitory computer-readable medium of claim 1, wherein the information processing apparatus is configured to collect and process facial data planar points from the person via the image processing apparatus.

10. A system configured to calculate vital signs comprising:
smart glasses configured to be worn on a user's head;
a camera coupled to the smart glasses configured to captures images of a person;
one or more sensors coupled to the smart glasses configured to measure associated signals from the person;
a computer configured to calculate and have displayed to the user vital signs of the person based on the captured images and/or signals.

11. The system of claim 10, further configured to permit the capture of the images and/or signals by the user via voice activation.

12. The system of claim 10, further configured to permit the calculation and display of vital signs via voice activation.

13. The system of claim 10, wherein the vital signs are of the person are displayed to the user via an augmented reality interface on the smart glasses.

14. The system of claim 10, wherein the vital signs to be calculated include body temperature, blood pressure, heart rate, O2 saturation, body mass index, age, or stress level.

15. The system of claim 10, wherein the vital signs to be calculated include body temperature, blood pressure, heart rate, O2 saturation, body mass index, age, or stress level.

16. The system of claim 10, wherein the calculation of vital signs is performed using the Anura™ software application by NuraLogix™ Corporation.

17. The system of claim 10, wherein the smart glasses includes a thermal camera.

18. The system of claim 10, wherein the information processing apparatus is configured to collect and process facial data planar points from the person via the image processing apparatus.

19. A method for calculating vital signs comprising:
capturing, using smart glasses coupled to a user's head, images of a person;
capturing, using one or more sensors coupled to the smart glasses, one or more associated signals from the person;
calculating vital signs of the person based on the images and/or signals; and
displaying the vital signs to the user.

20. The method of claim 20, further configured to permit the capture, calculation or display of the images and/or signals by the user via voice activation;
wherein the vital signs are of the person are displayed to the user via an augmented reality interface on the smart glasses; and
wherein the vital signs to be calculated include body temperature, blood pressure, heart rate, O2 saturation, body mass index, age, or stress level.
